(19) United States
(12) Patent Application Publication
(10) Pub. No.: US 2011/0009477 A1
(43) Pub. Date: Jan. 13, 2011

(54) OLIGONUCLEOTIDE-BASED COMPOUNDS AS INHIBITORS OF TOLL-LIKE RECEPTORS

(75) Inventors: Dong Yu, Westboro, MA (US); Lakshmi Bhagat, Framingham, MA (US); Daqing Wang, Bedford, MA (US); Ekambar Kandimalla, Southboro, MA (US); Sudhir Agrawal, Shrewsbury, MA (US)

Correspondence Address: PRETI, FLAHERTY, BELIVEAU & PACHIOS, LLP, PO Box 9546, One City Center, Portland, ME, 04112-9546, US

(73) Assignee: IDERA PHARMACEUTICALS, INC., Cambridge, MA (US)

(21) Appl. No.: 12/832,439

(22) Filed: Jul. 8, 2010

Related U.S. Application Data
(60) Provisional application No. 61/223,926, filed on Jul. 8, 2009.

(51) Int. Cl. A61K 31/7052; C07H 19/16; A61P 37/06

(52) U.S. Cl. 514/44 R; 536/23.1

(57) ABSTRACT

The invention provides novel oligonucleotide-based TLR antagonists containing a modified immune stimulatory motif and the use of such compounds in the prevention and treatment of TLR-medicated diseases. These oligonucleotide-based TLR antagonists containing a modified immune stimulatory motif have one or more chemical modifications in the immune stimulatory motif, which would be immune stimulatory but for the modification.

### BACKGROUND OF THE INVENTION

1. Field of the Invention

The invention generally relates to the field of immunology and immunotherapy, and more specifically to immune inhibitory oligonucleotide compositions containing a modified immune stimulatory motif, and optionally modifications to the nucleotides flanking the modified immune stimulatory motif, and their use for inhibiting and/or suppressing Toll-like Receptor-mediated immune responses.

2. Summary of the Related Art

Toll-like receptors (TLRs) are present on many cells of the immune system and have been shown to be involved in the innate immune response (Hornung, V. et al., (2002) J. Immunol. 168:4531-4537). In vertebrates, this family consists of ten proteins called TLR1 to TLR10, which are known to recognize pathogen associated molecular patterns from bacteria, fungi, parasites and viruses (Poltorak, a. et al. (1998) Science 282:2085-2088; Underhill, D. M., et al. (1999) Nature 401:811-815; Hayashi, F. et. al (2001) Nature 410:1099-1103; Zhang, D. et al. (2004) Science 303:1522-1526; Meier, A. et al. (2003) Cell. Microbiol. 5:561-570; Campos, M. A. et al. (2001) J. Immunol. 167: 416-423; Hoebe, K. et al. (2003) Nature 424: 743-748; Lund, J. (2003) J. Exp. Med. 198:513-520; Heil, F. et al. (2004) Science 303:1526-1529; Diebold, S. S., et al. (2004) Science 303:1529-1531; Hornung, V. et al. (2004) J. Immunol. 173:5935-5943). TLRs are a key means by which mammals recognize and mount an immune response to foreign molecules and also provide a means by which the innate and adaptive immune responses are linked (Akira, S. et al. (2001) Nature Immunol. 2:675-680; Medzhitov, R. (2001) Nature Rev. Immunol. 1:135-145). TLRs have also been shown to respond to damage associated molecular pattern molecules (DAMPs) (Song & Matzinger (2004) Nature Rev. Immunol. 4:469-478. These molecules are known to vary in their composition, with TLRs recognizing and responding to those DAMPs that contain DNA or RNA. TLRs have also been shown to play a role in the pathogenesis of many diseases, including autoimmunity, infectious disease, and inflammation (Cook, D. N. et al. (2004) Nature Immunol. 5:975-979) and the regulation of TLR-mediated activation using appropriate agents may provide a means for disease intervention.

Some TLRs are located on the cell surface to detect and initiate a response to extracellular pathogens and other TLRs are located inside the cell to detect and initiate a response to intracellular pathogens. Table 1 provides a representation of TLRs, the cell types containing the receptor and the known agonists therefore (Diebold, S. S. et al. (2004) Science 303:1529-1531; Liew, F. et al. (2005) Nature 5:446-458; Hemmi H et al. (2002) Nat Immunol 3:196-200; Jurk M et al., (2002) Nat Immunol 3:499; Lee J et al. (2003) Proc. Natl. Acad. Sci. USA 100:6646-6651); (Alexopoulou, L. (2001) Nature 413 :732-738).

TABLE 1

| TLR Molecule | Agonist | Cell Types Containing Receptor |
|---|---|---|
| Cell Surface TLRs: | | |
| TLR2 | bacterial lipopeptides | Monocytes/macrophages; |
| | | Myeloid dendritic cells; Mast cells |
| TLR4 | gram negative bacteria | Monocytes/macrophages; |
| | | Myeloid dendritic cells; Mast cells; |
| | | Intestinal epithelium |
| TLR5 | motile bacteria | Monocyte/macrophages; Dendritic |
| | | cells; Intestinal epithelium |
| TLR6 | gram positive bacteria | Monocytes/macrophages; Mast |
| | | cells; B lymphocytes |
| Endosomal TLRs: | | |
| TLR3 | double stranded RNA viruses | Dendritic cells; B lymphocytes |
| TLR7 | single stranded RNA viruses; | Monocytes/macrophages; |
| | RNA-immunoglobulin | Plasmacytoid dendritic cells; B |
| | complexes | lymphocytes |
| TLR8 | single stranded RNA viruses; | Monocytes/macrophages; |
| | RNA-immunoglobulin | Dendritic cells; Mast cells |
| | complexes | |
| TLR9 | DNA containing unmethylated | Monocytes/macrophages; |
| | “CpG” motifs; DNA- | Plasmacytoid dendritic cells; B |
| | immunoglobulin complexes | lymphocytes |

Certain unmethylated CpG motifs present in bacterial and synthetic DNA have been shown to activate the immune system and induce antitumor activity. (Tokunaga T et al., J. Natl. Cancer Inst. (1984) 72:955-962; Shimada S, et al., Jpn. H cancer Res, 1986, 77, 808-816; Yamamoto S, et al., Jpn. J. Cancer Res., 1986, 79, 866-73). Other studies using antisense oligonucleotides containing CpG dinucleotides have been shown to stimulate immune responses (Zhao Q, et al. (1996) Biochem. Pharmacol. 26:173-182). Subsequent studies demonstrated that TLR9 recognizes unmethylated CpG motifs present in bacterial and synthetic DNA (Hemmi, H. et al. (2000) Nature 408:740-745). Other modifications of CpG-containing phosphorothioate oligonucleotides can also affect their ability to act as stimulators of immune response through TLR9 (see, e.g., Zhao et al., Biochem. Pharmacol. (1996) 51:173-182; Zhao et al. (1996) Biochem Pharmacol. 52:1537-1544; Zhao et al. (1997) Antisense Nucleic Acid Drug Dev. 7:495-502; Zhao et al (1999) Bioorg. Med. Chem. Lett. 9:3453-3458; Zhao et al. (2000) Bioorg. Med. Chem. Lett. 10:1051-1054; Yu, D. et al. (2000) Bioorg. Med. Chem. Lett. 10:2585-2588; Yu, D. et al. (2001) Bioorg. Med. Chem. Lett. 11:2263-2267; and Kandimalla, E. et al. (2001) Bioorg. Med. Chem. 9:807-813). In addition, structure activity relationship studies have allowed identification of synthetic motifs and novel DNA-based compounds that induce specific immune response profiles that are distinct from those resulting from unmethylated CpG dinucleotides. (Kandimalla, E. et al. (2005) Proc. Natl. Acad. Sci. USA 102:6925-6930. Kandimalla, E. et al. (2003) Proc. Nat. Acad. Sci. USA 100:14303-14308; Cong, Y. et al. (2003) Biochem Biophys Res. Commun. 310:1133-1139; Kandimalla, E. et al. (2003) Biochem. Biophys. Res. Commun. 306:948-953; Kandimalla, E. et al. (2003) Nucleic Acids Res. 31:2393-2400; Yu, D. et al. (2003) Bioorg. Med. Chem. 11:459-464; Bhagat, L. et al. (2003) Biochem. Biophys. Res. Commun. 300:853-861; Yu, D. et al. (2002) Nucleic Acids Res.30:4460-4469; Yu, D. et al. (2002) J. Med. Chem. 45:4540-4548. Yu, D. et al. (2002) Biochem. Biophys. Res. Commun. 297:83-90; Kandimalla. E. et al. (2002) Bioconjug. Chem. 13:966-974; Yu, D. et al. (2002) Nucleic Acids Res. 30:1613-1619; Yu, D. et al. (2001) Bioorg. Med. Chem. 9:2803-2808; Yu, D. et al. (2001) Bioorg. Med. Chem. Lett. 11:2263-2267; Kandimalla, E. et al. (2001) Bioorg. Med. Chem. 9:807-813; Yu, D. et al. (2000) Bioorg. Med. Chem. Lett. 10:2585-2588; Putta, M. et al. (2006) Nucleic Acids Res. 34:3231-3238). It has been reported that certain nucleotide, backbone, and linker modifications which, upon site-specific incorporation in the flanking sequence 5′- or 3′- to the CpG dinucleotide, have significant influence on immune stimulatory activity (Yu, D. et. al (2002) Nuc. Acid Res. 30:1613-1619; Agrawal, S. et. al. (2001) Curr. Cancer. Drug Targets 1:197-209; Yu, D., et. al (2001) Bioorg. Med. Chem. 9:2803-2808; Yu, D. et. al (2001) Bioorg. Med. Chem Lett. 11:2263-2267; Yu, D. et. al (2003) Bioorg. Med. Chem. 11:459-464; Yu, D. et. al (2002) J. Med. Chem. 45-4540-4548; Zhao, Q. et. al (1999) Bioorg. Med. Chem. Lett. 9:3453-3458; Zhao, Q. et. al (2000) Bioorg. Med. Chem. Lett. 10:1051-1054). In addition, incorporation of 2′-O-methyl ribonucleotides in immune regulatory oligonucleotides in the first or second nucleotide position adjacent to the immune stimulatory dinucleotide on the 5′-side was reported to abrogate the immune stimulatory activity of the oligonucleotide and the presence of 2′-O-methylribonuclotide substitutions in the sequence flanking the immune stimulatory motif not only neutralize immune stimulatory activity but also caused the molecule to act as a TLR antagonist in vitro and in vivo (US20080089883; US20090060898; US20090087388; US20090081198).

The selective localization of TLRs and the signaling generated therefrom, provides some insight into the role of TLRs in the immune response. The immune response involves both an innate and an adaptive response based upon the subset of cells involved in the response. For example, the T helper (Th) cells involved in classical cell-mediated functions such as delayed-type hypersensitivity and activation of cytotoxic T lymphocytes (CTLs) are Th1 cells. This response is the body's innate response to antigens (e.g. viral infections, intracellular pathogens, and tumor cells), and results in a secretion of IFN-gamma and a concomitant activation of CTLs. Alternatively, the Th cells involved as helper cells for B-cell activation are Th2 cells. Th2 cells have been shown to be activated in response to bacteria and parasites and may mediate the body's adaptive immune response (e.g. IgE production and eosinophil activation) through the secretion of IL-4 and IL-5. The type of immune response is influenced by the cytokines produced in response to antigen exposure and the differences in the cytokines secreted by Th1 and Th2 cells may be the result of the different biological functions of these two subsets.

As a result of their involvement in regulating an inflammatory response, TLRs have been shown to play a role in the pathogenesis of many diseases, including autoimmunity, infectious disease and inflammation (Papadimitraki et al. (2007) J. Autoimmun. 29: 310-318; Sun et al. (2007) Inflam. Allergy Drug Targets 6:223-235; Diebold (2008) Adv. Drug Deliv. Rev. 60:813-823; Cook, D. N. et al. (2004) Nature Immunol. 5:975-979; Tse and Horner (2008) Semin. Immunopathol. 30:53-62; Tobias & Curtiss (2008) Semin. Immunopathol. 30:23-27; Ropert et al. (2008) Semin. Immunopathol. 30:41-51; Lee et al. (2008) Semin. Immunopathol. 30:3-9; Gao et al. (2008) Semin. Immunopathol. 30:29-40; Vijay-Kumar et al. (2008) Semin. Immunopathol. 30:11-21). While activation of TLRs is involved in mounting an immune response, an uncontrolled stimulation of the immune system through TLRs may exacerbate certain diseases. In recent years, several groups have shown the use of natural or synthetic oligodeoxyoligonucleotides (ODNs) as inhibitors of inflammatory cytokines (Lenert, P. et al. (2003) DNA Cell Biol. 22(10):621-631).

Krieg et al. (US2007/0202128) reported using oligonucleotides that are complimentary to certain targeted sequences and that (i) do not contain CG dinucleotides or (ii) that contain CG dinucleotides where the C is 5-MethylC, to compete for binding with oligonucleotides containing non-methylated CG dinucleotides. However, other studies have shown that such oligonucleotides lacking CG motifs or having a methyl CG motif are merely inactive. In addition, using certain synthetic ODNs, Lenert et al. report the ability to produce inhibitory ODNs (Lenert, P. et al. (2003) DNA Cell Biol. 22(10):621-631). These inhibitory ODN require two triplet sequences, a proximal “CCT” triplet and a distal “GGG” triplet. In addition to these triplet-containing inhibitory ODNs, several groups have shown other specific DNA sequences that could inhibit TLR-9-mediated activation by CpG-containing ODNs. These “inhibitory” or “suppressive” motifs are rich in “G” (e.g. “GGG” or “GGGG”) or “GC” sequences, tend to be methylated, and are present in the DNA of mammals and certain viruses (see e.g.; Chen, Y., et al., Gene Ther. 8: 1024-1032 (2001); Stunz, L. L., Eur. J. Immunol. 32: 1212-1222 (2002)). Duramad, O., et al., J. Immunol., 174: 5193-5200 (2005) and Jurk et. al (US 2005/0239733), describe a structure for inhibitory DNA oligonucleotides containing a GGGG motif within the sequences. Patole et al. demonstrate that GGGG containing ODNs will suppress systemic lupus (Patole, P. et al. (2005) J. Am. Soc. Nephrol. 16:3273-3280). Additionally, Gursel, I., et al., J. Immunol., 171: 1393-1400 (2003), describe repetitive TTAGGG elements, which are present at high frequency in mammalian telomeres, down-regulate CpG-induced immune activation. Shirota, H., et al., J. Immunol., 173: 5002-5007 (2004), demonstrate that synthetic oligonucleotides containing the TTAGGG element mimic this activity and could be effective in the prevention/treatment of certain Th1-dependent autoimmune diseases.

In contrast, several studies have called into question the view that poly G containing ODNs are acting as antagonists of TLRs. For example, U.S. Pat. No. 6,426,334, Agrawal et al., demonstrate that administering CpG oligonucleotides containing GGGG strings have potent antiviral and anticancer activity, and further that administration of these compounds will cause an increase in serum IL-12 concentration. In addition, CpG oligos containing polyG sequences are known to induce immune responses through TLR9 activation (Verthelyi D et al, J Immunol. 166, 2372, 2001; Gursel M et al, J Leukoc Biol, 71, 813, 2001, Krug A et al, Eur J Immunol, 31, 2154, 2001) and show antitumor and antiviral activities (Ballas G K et al, J Immunol, 167, 4878, 2001; Verthelyi D et al, J Immunol, 170, 4717, 2003). In addition, polyG oligonucleotides are also known to inhibit HIV and Rel A (McShan W M, et al, J Biol Chem., 267(8):5712-21, 1992; Rando, R F et al., J Biol Chem, 270(4):1754-60, 1995; Benimetskaya L, et al., Nucleic Acids Res., 25(13):2648-56, 1997). Also, ODNs containing an immune stimulatory CpG motif and 4 consecutive G nucleotides (class A ODNs) induce interferon-α production and a Th1 shift in the immune response. Moreover, in preclinical disease models, Class A ODN have been shown to induce a TLR-mediated immune response. Further, oligonucleotides containing guanosine strings have been shown to form tetraplex structures, act as aptamers, and inhibit thrombin activity (Bock L C et al., Nature, 355:564-6, 1992; Padmanabhan, K et al., J Biol Chem., 268(24):17651-4, 1993).

Thus, there remains a need to identify immune inhibitory oligonucleotides that are effective antagonist of TLRs.

### BRIEF SUMMARY OF THE INVENTION

The invention provides novel immune regulatory oligonucleotide-based TLR antagonists containing a modified immune stimulatory motif and methods of use thereof. These compounds have one or more chemical modifications in the immune stimulatory motif, which would be immune stimulatory but for the modification.

The oligonucleotide-based TLR antagonists containing a modified immune stimulatory motif, according to the invention, have the structure 5-Nₘ-N₃N₂N₁CGN¹N²N³-N^{m}-3′, wherein CG is the modified immune stimulatory motif and C is cytosine, or a pyrimidine nucleotide derivative selected from 5-methyl-dC, 2′-O-substituted-C, 2′-O-methyl-C, 2′-O-methoxyethyl-C, 2′-O-methoxyethyl-5-methyl-C, and 2′-O-methyl-5-methyl-C, and G is guanosine or a purine nucleotide derivative selected from 2′-O-substituted-G, 2′-O-methyl-G, and 2′-O-methoxyethyl-G; N₁-N₃ and N¹-N³, at each occurrence, is independently a nucleotide, nucleotide derivative or non-nucleotide linkage; Nₘ and N^{m}, at each occurrence, is independently a nucleotide, nucleotide derivative or non-nucleotide linkage; provided that at least one C and/or G of the modified immune stimulatory motif is a nucleotide derivative specified above; and optionally containing less than 3 consecutive guanosine nucleotides; wherein the modified immune stimulatory motif would be immune stimulatory but for the nucleotide derivative; and wherein m is a number from 0 to about 30.

In further embodiments of this aspect of the invention, the oligonucleotide-based TLR antagonist containing a modified immune stimulatory motif comprises one or more modified immune stimulatory motifs, wherein CG is the modified immune stimulatory motif and C is cytosine, or a pyrimidine nucleotide derivative selected from 5-methyl-dC, 2′-O-substituted-C, 2′-O-methyl-C, 2′-O-methoxyethoxy-C, 2′-O-methoxyethyl-5-methyl-C, 2′-O-methyl-5-methyl-C, and G is guanosine or a purine nucleotide derivative selected from 2′-O-substituted-G, 2′-O-methyl-G, and 2′-O-methoxyethoxy-G; provided that at least one C and/or G of the modified immune stimulatory motif is a nucleotide derivative specified above; and optionally containing less than 3 consecutive guanosine nucleotides; wherein the modified immune stimulatory motif would be immune stimulatory but for the nucleotide derivative.

In certain embodiments of the invention, oligonucleotide-based TLR antagonist containing a modified immune stimulatory motif may comprise at least two oligonucleotide-based TLR antagonists containing a modified immune stimulatory motif covalently linked by a nucleotide linkage, or a non-nucleotide linker, at their 5′-, 3′- or 2′-ends or by functionalized sugar or by functionalized nucleobase via a non-nucleotide linker or a nucleotide linkage. Such oligonucleotide-based TLR antagonist containing a modified immune stimulatory motif may be branched. As a non-limiting example, the linker may be attached to the 3′-hydroxyl of a nucleotide. In such embodiments, the linker comprises a functional group, which is attached to the 3′-hydroxyl by means of a phosphate-based linkage like, for example, phosphodiester, phosphorothioate, phosphorodithioate, methylphosphonate, or by non-phosphate-based linkages.

The invention further provides a method for therapeutically treating a mammal having a disease mediated by a TLR, such method comprising administering to the mammal an oligonucleotide-based TLR antagonist containing a modified immune stimulatory motif compound in a pharmaceutically effective amount. In preferred embodiments, the disease is cancer, an autoimmune disorder, airway inflammation, inflammatory disorders, infectious disease, malaria, Lyme disease, ocular infections, conjunctivitis, skin disorders, psoriasis, scleroderma, cardiovascular disease, atherosclerosis, chronic fatigue syndrome, sarcoidosis, transplant rejection, allergy, asthma or a inflammation caused by a pathogen. Preferred autoimmune disorders include without limitation lupus erythematosus, multiple sclerosis, type I diabetes mellitus, irritable bowl syndrome, Chron's disease, rheumatoid arthritis, septic shock, alopecia universalis, acute disseminated encephalomyelitis, Addison's disease, ankylosing spondylitis, antiphospholipid antibody syndrome, autoimmune hemolytic anemia, autoimmune hepatitis, Bullous pemphigoid, chagas disease, chronic obstructive pulmonary disease, coeliac disease, dermatomyositis, endometriosis, Goodpasture's syndrome, Graves' disease, Guillain-Barré syndrome, Hashimoto's disease, hidradenitis suppurativa, idiopathic thrombocytopenic purpura, interstitial cystitis, morphea, myasthenia gravis, narcolepsy, neuromyotonia, pemphigus, pernicious anaemia, polymyositis, primary biliary cirrhosis, schizophrenia, Sjögren's syndrome, temporal arteritis (“giant cell arteritis”), vasculitis, vitiligo, vulvodynia and Wegener's granulomatosis. Preferred inflammatory disorders include without limitation airway inflammation, asthma, autoimmune diseases, chronic inflammation, chronic prostatitis, glomerulonephritis, Behçet's disease, hypersensitivities, inflammatory bowel disease, reperfusion injury, rheumatoid arthritis, transplant rejection, ulcerative colitis, uveitis, conjunctivitis, and vasculitis.

The invention further provides a method for preventing a disease mediated by a TLR in a mammal, such method comprising administering to the mammal an oligonucleotide-based TLR antagonist containing a modified immune stimulatory motif in a pharmaceutically effective amount. In preferred embodiments, the disease is cancer, an autoimmune disorder, airway inflammation, inflammatory disorders, infectious disease, malaria, Lyme disease, ocular infections, conjunctivitis, skin disorders, psoriasis, scleroderma, cardiovascular disease, atherosclerosis, chronic fatigue syndrome, sarcoidosis, transplant rejection, allergy, asthma or a inflammation caused by a pathogen. Preferred autoimmune disorders include without limitation lupus erythematosus, multiple sclerosis, type I diabetes mellitus, irritable bowl syndrome, Chron's disease, rheumatoid arthritis, septic shock, alopecia universalis, acute disseminated encephalomyelitis, Addison's disease, ankylosing spondylitis, antiphospholipid antibody syndrome, autoimmune hemolytic anemia, autoimmune hepatitis, Bullous pemphigoid, chagas disease, chronic obstructive pulmonary disease, coeliac disease, dermatomyositis, endometriosis, Goodpasture's syndrome, Graves' disease, Guillain-Barré syndrome, Hashimoto's disease, hidradenitis suppurativa, idiopathic thrombocytopenic purpura, interstitial cystitis, morphea, myasthenia gravis, narcolepsy, neuromyotonia, pemphigus, pernicious anaemia, polymyositis, primary biliary cirrhosis, schizophrenia, Sjögren's syndrome, temporal arteritis (“giant cell arteritis”), vasculitis, vitiligo, vulvodynia and Wegener's granulomatosis. Preferred inflammatory disorders include without limitation airway inflammation, asthma, autoimmune diseases, chronic inflammation, chronic prostatitis, glomerulonephritis, Behçet's disease, hypersensitivities, inflammatory bowel disease, reperfusion injury, rheumatoid arthritis, transplant rejection, ulcerative colitis, uveitis, conjunctivitis and vasculitis.

In some preferred embodiments, the oligonucleotide-based TLR antagonist containing a modified immune stimulatory motif is administered in combination with one or more vaccines, antigens, antibodies, cytotoxic agents, allergens, antibiotics, antisense oligonucleotides, TLR agonists, TLR antagonists, peptides, proteins, gene therapy vectors, DNA vaccines, adjuvants, kinase inhibitors, antiviral agents, antimalarial drugs, or co-stimulatory molecules or combinations thereof. In some preferred embodiments, the route of administration is parenteral, mucosal delivery, oral, sublingual, transdermal, topical, inhalation, intranasal, aerosol, intraocular, intratracheal, intrarectal, vaginal, by gene gun, dermal patch or in eye drop or mouthwash form.

### BRIEF DESCRIPTION OF THE DRAWINGS

FIG. 1 depicts the ability of exemplary oligonucleotide-based TLR antagonists containing a modified immune stimulatory motif (SEQ ID NOs 2-6) to inhibit TLR9 activity in HEK293 cells treated according to Example 2. The data demonstrate that at each dosage, the oligonucleotide-based TLR antagonists containing a modified immune stimulatory motif according to the invention inhibit the activity of the control TLR9 agonist (SEQ ID NO 1).

FIG. 2A depicts the inability of exemplary oligonucleotide-based TLR antagonists containing a modified immune stimulatory motif (SEQ ID NOs 2-6) to activate TLR9 and subsequently induce NF-κB in J774 cells treated according to Example 2. The data demonstrate that at each dosage, the oligonucleotide-based TLR antagonists containing a modified immune stimulatory motif according to the invention do not activate TLRs.

FIG. 2B depicts the ability of exemplary oligonucleotide-based TLR antagonists containing a modified immune stimulatory motif (SEQ ID NOs 2-6) to inhibit TLR9 activity in J774 cells treated according to Example 2. The data demonstrate that at each dosage, the oligonucleotide-based TLR antagonists containing a modified immune stimulatory motif according to the invention inhibit the activity of a TLR9 agonist (SEQ ID NO 1).

FIGS. 3A and 3B depict absence of TLR-mediated cytokine induction by oligonucleotide-based TLR antagonists containing a modified immune stimulatory motif (SEQ ID NOs 2-6) in mouse spleen cells treated according to Example 2. The data demonstrate that oligonucleotide-based TLR antagonists containing a modified immune stimulatory motif according to the invention do not induce IL-6 or IL-12 production. More generally, these data demonstrate that oligonucleotide-based TLR antagonists containing a modified immune stimulatory motif according to the invention do not activate TLRs.

FIGS. 4A and 4B depict inhibition of TLR-inhibitory properties of exemplary oligonucleotide-based TLR antagonists containing a modified immune stimulatory motif (SEQ ID NOs 2-6) in mouse spleen cells treated according to Example 2. The data demonstrate that oligonucleotide-based TLR antagonists containing a modified immune stimulatory motif according to the invention do not induce TLR activation and the subsequent cykokine production and that oligonucleotide-based TLR antagonists containing a modified immune stimulatory motif according to the invention inhibit activation of TLR9 by an agonist and the subsequent cytokine production.

FIGS. 4C and 4D depict dose dependent inhibition of TLR9 activation by exemplary oligonucleotide-based TLR antagonists containing a modified immune stimulatory motif according to the invention in mouse spleen cells treated according to Example 2. The data demonstrate that oligonucleotide-based TLR antagonists containing a modified immune stimulatory motif according to the invention can inhibit TLR9 stimulation and the subsequent cytokine production in a dose dependent fashion.

FIG. 5 depicts the in vivo TLR-inhibitory properties of exemplary oligonucleotide-based TLR antagonists containing a modified immune stimulatory motif (SEQ ID NOs 2-6) administered according to Example 3. The data demonstrate that oligonucleotide-based TLR antagonists containing a modified immune stimulatory motif according to the invention do not induce in vivo TLR activation and subsequent cytokine or chemokine production. The data further demonstrate that oligonucleotide-based TLR antagonists containing a modified immune stimulatory motif according to the invention inhibit in vivo TLR activation and subsequent cytokine and chemokine production by a TLR9 agonist (SEQ ID NO 1).

FIG. 6A depicts the in vivo TLR inhibitory activity of exemplary oligonucleotide-based TLR antagonists containing a modified immune stimulatory motif in mice treated according to Example 4. The data demonstrate that oligonucleotide-based TLR antagonists containing a modified immune stimulatory motif according to the invention can inhibit in vivo activation of an immune response by a TLR9 agonist in a dose-dependent fashion.

FIG. 6B depicts in vivo TLR inhibitory activity of exemplary oligonucleotide-based TLR antagonists containing a modified immune stimulatory motif in mice treated according to Example 5. The data demonstrate that oligonucleotide-based TLR antagonists containing a modified immune stimulatory motif according to the invention can inhibit in vivo activation of an immune response by a TLR9 agonist and the activity is dependent on the dose of the TLR9 agonist.

FIG. 6C depicts the in vivo TLR inhibitory activity of exemplary oligonucleotide-based TLR antagonists containing a modified immune stimulatory motif in mice treated according to Example 4. The data demonstrate that oligonucleotide-based TLR antagonists containing a modified immune stimulatory motif according to the invention can inhibit in vivo TLR activation by a TLR agonist (SEQ ID NO 12).

FIG. 7 depicts the duration of in vivo TLR inhibitory activity of exemplary oligonucleotide-based TLR antagonists containing a modified immune stimulatory motif in mice treated according to Example 6. The data demonstrate that oligonucleotide-based TLR antagonists containing a modified immune stimulatory motif can inhibit in vivo TLR stimulation for a sustained period of time.

FIG. 8 depicts the in vivo specificity of exemplary oligonucleotide-based TLR antagonists containing a modified immune stimulatory motif in mice treated according to Example 7. The data demonstrate that oligonucleotide-based TLR antagonists containing a modified immune stimulatory motif according to the invention selectively inhibit the activity of TLR7 and TLR9.

### DETAILED DESCRIPTION OF THE PREFERRED EMBODIMENTS

The present invention relates to the therapeutic use of novel oligonucleotides as immune modulatory agents for immunotherapy applications. Specifically, the invention provides oligonucleotide-based TLR antagonists containing a modified immune stimulatory motif. These compounds act as antagonists of toll-like receptors (TLRs) to inhibit and/or suppress a TLR-mediated immune response. These compounds have unique sequences that inhibit or suppress TLR-mediated signaling in response to endogenous and/or exogenous TLR ligands or agonists. The references cited herein reflect the level of knowledge in the field and are hereby incorporated by reference in their entirety. Any conflicts between the teachings of the cited references and this specification shall be resolved in favor of the latter.

The invention provides compounds and methods for suppressing an immune response caused by TLRs and can be used for immunotherapy applications such as, but not limited to, treatment of cancer, autoimmune disorders, asthma, respiratory allergies, food allergies, skin allergies, systemic lupus erythematosus (SLE), arthritis, pleurisy, chronic infections, inflammatory diseases, inflammatory bowl syndrome, sepsis, and bacteria, parasitic, and viral infections in adult and pediatric human and veterinary applications. Thus, the invention further provides oligonucleotide-based TLR antagonists containing a modified immune stimulatory motif having optimal levels of immune inhibitory activity for immunotherapy and methods for making and using such compounds. In addition, oligonucleotide-based TLR antagonists containing a modified immune stimulatory motif of the invention are useful in combination with, for example, DNA vaccines, antigens, antibodies, antiviral agents, antimalarial drugs (for example, chloroquine and hydroxychloroquine) and allergens; and in combination with chemotherapeutic agents (both chemotherapies and targeted therapies) and/or antisense oligonucleotides for prevention and treatment of diseases.

The term “oligonucleotide” generally refers to a polynucleoside comprising a plurality of linked nucleoside units. Such oligonucleotides can be obtained from existing nucleic acid sources, including genomic or cDNA, but are preferably produced by synthetic methods. In preferred embodiments each nucleoside unit can encompass various chemical modifications and substitutions as compared to wild-type oligonucleotides, including but not limited to modified nucleoside base and/or modified sugar unit(s). Examples of chemical modifications are known to the person skilled in the art and are described, for example, in Uhlmann E et al. (1990) Chem. Rev. 90:543; “Protocols for Oligonucleotides and Analogs” Synthesis and Properties & Synthesis and Analytical Techniques, S. Agrawal, Ed, Humana Press, Totowa, USA 1993; and Hunziker, J. et al. (1995) Mod. Syn. Methods 7:331-417; and Crooke, S. et al. (1996) Ann. Rev. Pharm. Tox. 36:107-129. The nucleoside residues can be coupled to each other by any of the numerous known internucleoside linkages. Such internucleoside linkages include, without limitation, phosphodiester, phosphorothioate, phosphorodithioate, alkylphosphonate, alkylphosphonothioate, phosphotriester, phosphoramidate, siloxane, carbonate, carboalkoxy, acetamidate, carbamate, morpholino, borano, thioether, bridged phosphoramidate, bridged methylene phosphonate, bridged phosphorothioate, and sulfone internucleoside linkages. The term “oligonucleotide” also encompasses polynucleosides having one or more stereospecific internucleoside linkage (e.g., (R_{P})- or (S_{P})-phosphorothioate, alkylphosphonate, or phosphotriester linkages). As used herein, the terms “oligonucleotide” and “dinucleotide” are expressly intended to include polynucleosides and dinucleosides having any such internucleoside linkage, whether or not the linkage comprises a phosphate group. In certain preferred embodiments, these internucleoside linkages may be phosphodiester, phosphorothioate or phosphorodithioate linkages, or combinations thereof.

The term “2′-substituted” generally includes nucleosides in which the hydroxyl group at the 2′ position of the pentose moiety is substituted to produce a 2′-substituted or 2′-O-substituted nucleoside. In certain embodiments, such substitution is with a lower hydrocarbyl group containing 1-6 saturated or unsaturated carbon atoms, with a halogen atom, or with an aryl group having 6-10 carbon atoms, wherein such hydrocarbyl, or aryl group may be unsubstituted or may be substituted, e.g., with halo, hydroxy, trifluoromethyl, cyano, nitro, acyl, acyloxy, alkoxy, carboxyl, carboalkoxy, or amino groups. Non limiting examples of 2′-O-substituted nucleosides include, without limitation 2′-amino, 2′-fluoro, 2′-allyl, 2′-O-alkyl and 2′-propargyl nucleosides, 2′-O-methylnucleosides and 2′-O-methoxyethoxynucleosides.

The term “3′”, when used directionally, generally refers to a region or position in a polynucleotide or oligonucleotide 3′ (downstream) from another region or position in the same polynucleotide or oligonucleotide.

The term “5′”, when used directionally, generally refers to a region or position in a polynucleotide or oligonucleotide 5′ (upstream) from another region or position in the same polynucleotide or oligonucleotide.

The term “about” generally means that the exact number is not critical. Thus, the number of nucleoside residues in the oligonucleotides is not critical, and oligonucleotides having one or two fewer nucleoside residues, or from one to several additional nucleoside residues are contemplated as equivalents of each of the embodiments described above.

The term “agonist” generally refers to a substance that binds to a receptor of a cell and induces a response. Such response may be an increase in the activity mediated by the receptor. An agonist often mimics the action of a naturally occurring substance such as a ligand.

The term “antagonist” generally refers to a substance that can bind to a receptor, but does not produce a biological response upon binding. The antagonist can block, inhibit or attenuate the response mediated by an agonist or ligand and may compete with agonist for binding to a receptor. Such antagonist activity may be reversible or irreversible.

The term “adjuvant” generally refers to a substance which, when added to an immunogenic agent such as vaccine or antigen, enhances or potentiates an immune response to the agent in the recipient host upon exposure to the mixture.

The term “airway inflammation” generally includes, without limitation, asthma.

The term “allergen” generally refers to an antigen or antigenic portion of a molecule, usually a protein, which elicits an allergic response upon exposure to a subject. Typically the subject is allergic to the allergen as indicated, for instance, by the wheal and flare test or any method known in the art. A molecule is said to be an allergen even if only a small subset of subjects exhibit an allergic immune response upon exposure to the molecule.

The term “allergy” generally refers to an inappropriate immune response characterized by inflammation and includes, without limitation, food allergies and respiratory allergies.

The term “antigen” generally refers to a substance that is recognized and selectively bound by an antibody or by a T cell antigen receptor, resulting in induction of an immune response. Antigens may include but are not limited to peptides, proteins, nucleosides, nucleotides, nucleic acids, carbohydrates, lipids, and combinations thereof Antigens may be natural or synthetic and generally induce an immune response that is specific for that antigen.

The term “antiviral agent” generally refers to an agent that has the capacity to kill viruses, suppress their replication, cell binding or other essential functions and, hence, inhibits their capacity to multiply and reproduce. Such agents may act by stimulating cellular defenses against viruses.

The term “autoimmune disorder” generally refers to disorders in which “self” components undergo attack by the immune system.

The term “physiologically acceptable” generally refers to a material that does not interfere with the effectiveness of an oligonucleotide-based TLR antagonist containing a modified immune stimulatory motif compound and that is compatible with a biological system such as a cell, cell culture, tissue, or organism. Preferably, the biological system is a living organism, such as a mammal.

The term “carrier” generally encompasses any excipient, diluent, filler, salt, buffer, stabilizer, solubilizer, oil, lipid, lipid containing vesicle, microspheres, liposomal encapsulation, or other material well known in the art for use in pharmaceutical formulations. It will be understood that the characteristics of the carrier, excipient, or diluent will depend on the route of administration for a particular application. The preparation of pharmaceutically acceptable formulations containing these materials is described in, e.g., Remington's Pharmaceutical Sciences, 18th Edition, ed. A. Gennaro, Mack Publishing Co., Easton, Pa., 1990.

The term “co-administration” generally refers to the administration of at least two different substances sufficiently close in time to modulate an immune response. Co-administration refers to simultaneous administration, as well as temporally spaced order of up to several days apart, of at least two different substances in any order, either in a single dose or separate doses.

The term “disease or disorder mediated by a TLR” is intended to mean a condition having signs or symptoms that are contributed to, in whole or in part, by activation of a TLR.

The term an “effective amount” or a “sufficient amount” generally refers to an amount sufficient to affect a desired biological effect, such as beneficial results. Thus, an “effective amount” or “sufficient amount” will depend upon the context in which it is being administered. In the context of therapeutically treating a disease, an effective amount is an amount that ameliorates one or more sign or symptom of the disease. In the context of prophylactically preventing a disease, an effective amount is an amount that prevents or reduces the development of one or more sign or symptom of the disease. In the context of administering a composition that modulates an immune response to a co-administered antigen, an effective amount of an oligonucleotide-based TLR antagonist containing a modified immune stimulatory motif compound and antigen is an amount sufficient to achieve the desired modulation as compared to the immune response obtained when the antigen is administered alone. An effective amount may be administered in one or more administrations.

The term “in combination with” generally means in the course of treating a disease or disorder in a patient, administering an oligonucleotide-based TLR antagonist containing a modified immune stimulatory motif compound and an agent useful for treating the disease or disorder that does not diminish the immune modulatory effect of the oligonucleotide-based TLR antagonist containing a modified immune stimulatory motif compound. Such combination treatment may also include more than a single administration of an oligonucleotide-based TLR antagonist containing a modified immune stimulatory motif compound and/or independently an agent. The administration of the oligonucleotide-based TLR antagonist containing a modified immune stimulatory motif compound and/or the agent may be by the same or different routes.

The term “individual” or “subject” or “mammal” generally refers to but is not limited to, humans, non-human primates, rats, mice, cats, dogs, horses, cattle, cows, pigs, sheep, and rabbits.

The term “kinase inhibitor” generally refers to molecules that antagonize or inhibit phosphorylation-dependent cell signaling and/or growth pathways in a cell. Kinase inhibitors may be naturally occurring or synthetic and include small molecules that have the potential to be administered as oral therapeutics. Kinase inhibitors have the ability to rapidly and specifically inhibit the activation of the target kinase molecules. Protein kinases are attractive drug targets, in part because they regulate a wide variety of signaling and growth pathways and include many different proteins. As such, they have great potential in the treatment of diseases involving kinase signaling, including cancer, cardiovascular disease, inflammatory disorders, diabetes, macular degeneration and neurological disorders. A non-limiting example of a kinase inhibitor is sorafenib.

The term “nucleoside” generally refers to compounds consisting of a sugar, usually ribose or deoxyribose, and a purine or pyrimidine base.

The term “nucleotide” generally refers to a nucleoside comprising a phosphorous-containing group attached to the sugar.

As used herein, the term “pyrimidine nucleoside” refers to a nucleoside wherein the base component of the nucleoside is a pyrimidine base (e.g., cytosine (C) or thymine (T) or Uracil (U)). Similarly, the term “purine nucleoside” refers to a nucleoside wherein the base component of the nucleoside is a purine base (e.g., adenine (A) or guanine (G)).

The terms “analog” or “derivative” can be used interchangeably to generally refer to any purine and/or pyrimidine nucleotide or nucleoside that has a modified base and/or sugar. A modified base is a base that is not guanine, cytosine, adenine, thymine or uracil. A modified sugar is any sugar that is not ribose or 2′-deoxyribose and can be used in the backbone for an oligonucleotide.

The term “inhibiting” or “suppressing” generally refers to a decrease in a response or qualitative difference in a response, which could otherwise arise from eliciting and/or stimulation of a response.

The term “non-nucleotide linker” generally refers to any chemical moiety that can link two or more oligonucleotides other than through a phosphorous-containing or non-phosphorus linkage. Preferably such linker is from about 2 angstroms to about 200 angstroms in length.

The term “nucleotide linkage” generally refers to a 3′-5′ linkage that directly connects the 3′ and 5′ hydroxyl groups of two nucleosides through a phosphorous-containing linkage.

The terms “oligonucleotide motif” generally refers to an oligonucleotide sequence, including a dinucleotide. An “oligonucleotide motif that would be immune stimulatory, but for one or more modifications” means an oligonucleotide motif which is immune stimulatory in a parent oligonucleotide, but not in a derivative oligonucleotide, wherein the derivative oligonucleotide is based upon the parent oligonucleotide, but has one or more modifications to the oligonucleotide motif that reduce or eliminate immune stimulation.

The term “treatment” generally refers to an approach intended to obtain a beneficial or desired result, which may include alleviation of symptoms, or delaying or ameliorating a disease progression.

In a first aspect, the invention provides oligonucleotide-based TLR antagonists containing a modified immune stimulatory motif The term “oligonucleotide-based TLR antagonists containing a modified immune stimulatory motif” refers to an oligonucleotide compound that is an antagonist for one or more TLR, wherein the compound comprises one or more modified immune stimulatory motifs, wherein CG is the modified immune stimulatory motif and C is cytosine, or a pyrimidine nucleotide derivative selected from 5-methyl-dC, 2′-O-substituted-C, 2′-O-methyl-C, 2′-O-methoxyethyl-C, 2′-O-methoxyethyl-5-methyl-C, 2′-O-methyl-5-methyl-C, and G is guanosine or a purine nucleotide derivative selected from 2′-O-substituted-G, 2′-O-methyl-G, and 2′-O-methoxyethyl-G; provided that at least one C and/or G of the modified immune stimulatory motif is a nucleotide derivative specified above; and optionally containing less than 3 consecutive guanosine nucleotides; wherein the modified immune stimulatory motif would be immune stimulatory but for the nucleotide derivative replacing cytosine and/or guanosine. The oligonucleotide-based TLR antagonist containing a modified immune stimulatory motif compound may contain one or more additional modifications that enhances the inhibitory activity of the compound. Such modifications may be in the sequence flanking the modified immune stimulatory motif. Such modifications can be to the bases, sugar residues and/or the phosphate backbone of the nucleotides/nucleosides flanking the modified immune stimulatory motif or within the modified immune stimulatory motif. These modifications result in oligonucleotide-based TLR antagonists containing a modified immune stimulatory motif that suppresses TLR-mediated immune stimulation.

In preferred embodiments the oligonucleotide-based TLR antagonists containing a modified immune stimulatory motif is not an antisense oligonucleotide.

The general structure of the oligonucleotide-based TLR antagonists containing a modified immune stimulatory motif may be represented as 5′-Nₘ-N₃N₂N₁CGN¹N²N³-N^{m}-3′ wherein CG is the modified immune stimulatory motif and C is cytosine, or a pyrimidine nucleotide derivative selected from 5-methyl-dC, 2′-O-substituted-C, 2′-O-methyl-C, 2′-O-methoxyethoxy-C, 2′-O-methoxyethyl-5-methyl-C, and 2′-O-methyl-5-methyl-C, and G is guanosine or a purine nucleotide derivative selected from 2′-O-substituted-G, 2′-O-methyl-G, and 2′-O-methoxyethoxy-G; N₁-N₃ and N¹-N³, at each occurrence, is independently a nucleotide, nucleotide derivative or non-nucleotide linkage; Nₘ and N^{m}, at each occurrence, is independently a nucleotide, nucleotide derivative or non-nucleotide linkage; provided that at least one C and/or G of the modified immune stimulatory motif is a nucleotide derivative specified above; and optionally containing less than 3 consecutive guanosine nucleotides; wherein the modified immune stimulatory motif would be immune stimulatory but for the nucleotide derivative replacing cytosine and/or guanosine; and wherein m is a number from 0 to about 30.

In further embodiments of this aspect of the invention, the oligonucleotide-based TLR antagonist containing a modified immune stimulatory motif comprises one or more modified immune stimulatory motifs, wherein CG is the modified immune stimulatory motif and C is cytosine, or a pyrimidine nucleotide derivative selected from 5-methyl-dC, 2′-O-substituted-C, 2′-O-methyl-C, 2′-O-methoxyethoxy-C, 2′-O-methoxyethyl-5-methyl-C, and 2′-O-methyl-5-methyl-C, and G is guanosine or a purine nucleotide derivative selected from 2′-O-substituted-G, 2′-O-methyl-G, and 2′-O-methoxyethoxy-G; provided that at least one C and/or G of the modified immune stimulatory motif is a nucleotide derivative specified above; and optionally containing less than 3 consecutive guanosine nucleotides; wherein the modified immune stimulatory motif would be immune stimulatory but for the nucleotide derivative.

In certain embodiments of the invention, oligonucleotide-based TLR antagonist containing a modified immune stimulatory motif may comprise at least two oligonucleotide-based TLR antagonists containing a modified immune stimulatory motif covalently linked by a nucleotide linkage (“directly linked”), or a non-nucleotide linker, at their 5′-, 3′- or 2′-ends or by functionalized sugar or by functionalized nucleobase via a non-nucleotide linker or a nucleotide linkage. Such oligonucleotide-based TLR antagonist containing a modified immune stimulatory motif may be branched. As a non-limiting example, the linker may be attached to the 3′-hydroxyl of a nucleotide. In such embodiments, the linker comprises a functional group, which is attached to the 3′-hydroxyl by means of a phosphate-based linkage like, for example, phosphodiester, phosphorothioate, phosphorodithioate, methylphosphonate, or by non-phosphate-based linkages. Possible sites of conjugation for the ribonucleotide are indicated in Formula I, below, wherein B represents a heterocyclic base and wherein the arrow pointing to P indicates any attachment to phosphorous.

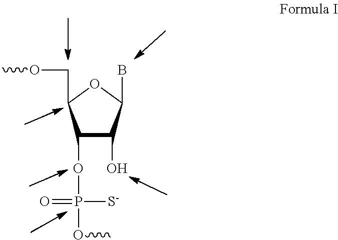

In some embodiments, the non-nucleotide linker is a small molecule, macromolecule or biomolecule, including, without limitation, polypeptides, antibodies, lipids, antigens, allergens, and oligosaccharides. In some other embodiments, the non-nucleotidic linker is a small molecule. For purposes of the invention, a small molecule is an organic moiety having a molecular weight of less than 1,000 Da. In some embodiments, the small molecule has a molecular weight of less than 750 Da.

In some embodiments, the small molecule is an aliphatic or aromatic hydrocarbon, either of which optionally can include, either in the molecular chain connecting the oligoribonucleotides or appended to it, one or more functional groups including, but not limited to, hydroxy, amino, thiol, thioether, ether, amide, thioamide, ester, urea, or thiourea. The small molecule can be cyclic or acyclic. Examples of small molecule linkers include, but are not limited to, amino acids, carbohydrates, cyclodextrins, adamantane, cholesterol, haptens and antibiotics. However, for purposes of describing the non-nucleotidic linker, the term “small molecule” is not intended to be a conventional 5′-3′ phosphorous-linked nucleotide.

In some embodiments, the non-nucleotidic linker is an alkyl linker or amino linker. The alkyl linker may be branched or unbranched, cyclic or acyclic, substituted or unsubstituted, saturated or unsaturated, chiral, achiral or racemic mixture. The alkyl linkers can have from about 2 to about 18 carbon atoms. In some embodiments such alkyl linkers have from about 3 to about 9 carbon atoms. Some alkyl linkers include one or more functional groups including, but not limited to, hydroxy, amino, thiol, thioether, ether, amide, thioamide, ester, urea, and thioether. Such alkyl linkers can include, but are not limited to, 1,2 propanediol, 1,2,3 propanetriol, 1,3 propanediol, triethylene glycol hexaethylene glycol, polyethylene glycol linkers (e.g. [—O—CH2-CH2-]ₙ (n=1-9)), methyl linkers, ethyl linkers, propyl linkers, butyl linkers, or hexyl linkers. In some embodiments, such alkyl linkers may include peptides or amino acids.

In some embodiments, the non-nucleotide linker may include, but are not limited to, those listed in Table 2.

TABLE 2
Representative Non-Nucleotidic Linkers

| Non-Nucleotidic | |
|---|---|
| Linker No. | Chemical Composition |
| 1 | Glycerol (1,2,3-Propanetriol) |
| 2 | 1,2,4, Butanetriol |
| 3 | 2-(hydroxymethyl)1,4-butanediol |
| 4 | 1,3,5-Pentanetriol |
| 5 | 1,1,1-Tris(hydroxymethyl)ethane |
| 6 | 1,1,1-Tris(hydroxymethyl)nitromethane |
| 7 | 1,1,1-Tris(hydroxymethyl)propane |
| 8 | 1,2,6-Methyl-1,3,5-pentanetriol |
| 9 | 1,2,3-Heptanetriol |
| 10 | 2-Amino-2-(hydroxymethyl)-1,3-propanediol |
| 11 | N-[Tris(hydroxymethyl)methyl]acrylamide |
| 12 | cis-1,3,5-Cyclohexanetriol |
| 13 | Cis-1,3,5-Tri(hydroxymethyl)cyclohexane |
| 14 | 1,3,5-Trihydroxyl-benzene |
| 15 | 3,5-Di(hydroxymethyl)benzene |
| 16 | 1,3-Di(hydroxyethoxy)-2-hydroxyl-propane |
| 17 | 1,3-Di(hydroxypropoxy)-2-hydroxyl-propane |
| 18 | 2-Deoxy-D-ribose |
| 19 | 1,2,4-Trihydroxyl-benzene |
| 20 | D-Galactoal |
| 21 | 1,6-anhydro-β-D-Glucose |
| 22 | 1,3,5-Tris(2-hydroxyethyl)-Cyanuric acid |
| 23 | Gallic acid |
| 24 | 3,5,7-Trihydroxyflavone |
| 25 | 4,6-Nitropyrogallol |
| 26 | Ethylene glycol |
| 27 | 1,3-Propanediol |
| 28 | 1,2-Propanediol |
| 29 | 1,4-Butanediol |
| 30 | 1,3-Butanediol |
| 31 | 2,3-Butanediol |
| 32 | 1,4-Butanediol |
| 33 | 1,5-Pentanediol |
| 34 | 2,4-Pentanediol |
| 35 | 1,6-Hexanediol |
| 36 | 1,2-Hexanediol |
| 37 | 1,5-Hexanediol |
| 38 | 2,5-Hexanediol |
| 39 | 1,7-Heptanediol |
| 40 | 1,8-Octanediol |
| 41 | 1,2-Octanediol |
| 42 | 1,9-Nonanediol |
| 43 | 1,12-Dodecanediol |
| 44 | Triethylene glycol |
| 45 | Tetraethylene glycol |
| 46 | Hexaethylene glycol |
| 47 | 2-(1-Aminopropyl)-1,3-propanediol |
| 48 | 1,2-Dideoxyribose |

In some embodiments, the small molecule linker is glycerol or a glycerol homolog of the formula HO—(CH₂)ₒ—CH(OH)—(CH₂)ₚ—OH, wherein o and p independently are integers from 1 to about 6, from 1 to about 4, or from 1 to about 3. In some other embodiments, the small molecule linker is a derivative of 1,3-diamino-2-hydroxypropane. Some such derivatives have the formula HO—(CH₂)ₘ—C(O)NH—CH₂—CH(OH)—CH₂—NHC(O)—(CH₂)ₘ—OH, wherein m is an integer from 0 to about 10, from 0 to about 6, from 2 to about 6, or from 2 to about 4

Some non-nucleotide linkers according to the invention permit attachment of more than two oligonucleotide-based TLR antagonists containing a modified immune stimulatory motif. For example, the small molecule linker glycerol has three hydroxyl groups to which oligonucleotide-based TLR antagonists containing a modified immune stimulatory motif may be covalently attached. Such compounds, therefore, comprise two or more oligonucleotide-based TLR antagonists containing a modified immune stimulatory motif linked to a nucleotide or a non-nucleotide linker, and can be referred to as being “branched”.

Oligonucleotide-based TLR antagonists containing a modified immune stimulatory motif may comprise at least two oligonucleotide-based TLR antagonists containing a modified immune stimulatory motif non-covalently linked, such as by electrostatic interactions, hydrophobic interactions, it-stacking interactions, hydrogen bonding and combinations thereof. Non-limiting examples of such non-covalent linkage includes Watson-Crick base pairing, Hoogsteen base pairing and base stacking Some of the ways in which two or more oligonucleotide-based TLR antagonists containing a modified immune stimulatory motif can be linked are shown in Table 3.

TABLE 3
Oligoribonucleotide Formulas II-X

| | |
|---|---|
| Formula II | 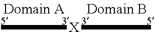 |
| Formula III | 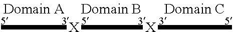 |
| Formula IV | 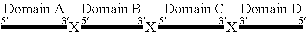 |
| Formula V | 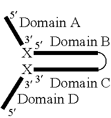 |
| Formula VI | 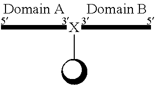 |
| Formula VII | 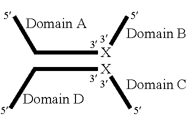 |
| Formula VIII | 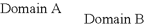 |
| Formula X | 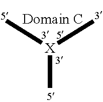 |
| Formula IX | 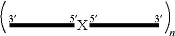 |
| Formula X | 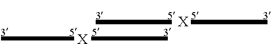 |

In certain embodiments, pyrimidine nucleosides in the immune regulatory oligonucleotides used in the compositions and methods according to the invention have the structure (II):

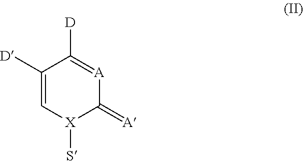

wherein:

D is a hydrogen bond donor;

D′ is selected from the group consisting of hydrogen, hydrogen bond donor, hydrogen bond acceptor, hydrophilic group, hydrophobic group, electron withdrawing group and electron donating group;

A is a hydrogen bond acceptor or a hydrophilic group;

A′ is selected from the group consisting of hydrogen bond acceptor, hydrophilic group, hydrophobic group, electron withdrawing group and electron donating group;

X is carbon or nitrogen; and

S′ is a pentose or hexose sugar ring, or a sugar analog.

In certain preferred embodiments, the pentose sugar is ribose or deoxyribose.

In certain preferred embodiments, the hexose sugar ring is glucose or fructose.

In certain preferred embodiments, the sugar analog is arabinose.

In certain embodiments, the sugar ring is derivatized with a phosphate moiety, modified phosphate moiety, or other linker moiety suitable for linking the pyrimidine nucleoside to another nucleoside or nucleoside analog.

In some embodiments hydrogen bond donors include, without limitation, —NH—, —NH₂, —SH and —OH. Preferred hydrogen bond acceptors include, without limitation, C═O, C═S, and the ring nitrogen atoms of an aromatic heterocycle, e.g., N3 of cytosine.

In some embodiments, (II) is a pyrimidine nucleoside derivative. Examples of pyrimidine nucleoside derivatives include, without limitation, 5-methyl-dC, 2′-O-substituted-C, 2′-O-methyl-C, 2′-O-methoxyethoxy-C, 2′-O-methoxyethyl-5-methyl-C, 2′-O-methyl-5-methyl-C, 5-hydroxycytosine, 5-hydroxymethylcytosine, N4-alkylcytosine, or N4-ethylcytosine, ara-C, 5-OH-dC, N3-Me-dC, and 4-thiouracil. Chemical modified derivatives also include, but are not limited to, thymine or uracil analogues. In some embodiments, the sugar moiety S′ in (II) is a sugar derivative. Suitable sugar derivatives include, but are not limited to, trehalose or trehalose derivatives, hexose or hexose derivatives, arabinose or arabinose derivatives.

In some embodiments, the purine nucleosides in immune regulatory oligonucleotides used in the compositions and methods according to the invention have the structure (III):

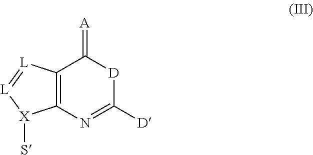

wherein:

D is a hydrogen bond donor;

D′ is selected from the group consisting of hydrogen, hydrogen bond donor, and hydrophilic group;

A is a hydrogen bond acceptor or a hydrophilic group;

X is carbon or nitrogen;

each L is independently selected from the group consisting of C, O, N and S; and

S′ is a pentose or hexose sugar ring, or a sugar analog (each as defined above).

In certain embodiments, the sugar ring is derivatized with a phosphate moiety, modified phosphate moiety, or other linker moiety suitable for linking the pyrimidine nucleoside to another nucleoside or nucleoside analog.

In certain embodiments hydrogen bond donors include, without limitation, —NH—, —NH₂, —SH and —OH. In certain embodiments hydrogen bond acceptors include, without limitation, C═O, C═S, —NO₂ and the ring nitrogen atoms of an aromatic heterocycle, e.g., N1 of guanine.

In some embodiments, (III) is a purine nucleoside derivative. Examples of purine nucleoside derivatives include, without limitation, guanine analogues such as 2′-O-substituted-G, 2′-O-methyl-G, 2′-O-methoxyethoxy-G, 7-deaza-G, 7-deaza-dG, ara-G, 6-thio-G, Inosine, Iso-G, loxoribine, TOG (7-thio-8-oxo)-G, 8-bromo-G, 8-hydroxy-G, 5-aminoformycin B, Oxoformycin, 7-methyl-G, 9-p-chlorophenyl-8-aza-G, 9-phenyl-G, 9-hexyl-guanine, 7-deaza-9-benzyl-G, 6-Chloro-7-deazaguanine, 6-methoxy-7-deazaguanine, 8-Aza-7-deaza-G(PPG), 2-(Dimethylamino)guanosine, 7-Methyl-6-thioguanosine, 8-Benzyloxyguanosine, 9-Deazaguanosine, 1-(B-D-ribofuranosyl)-2-oxo-7-deaza-8-methyl-purine, 1-(2′-deoxy-β-D-ribofuranosyl)-2-oxo-7-deaza-8-methyl-purine. Chemically modified derivatives also include, but are not limited to, adenine analogues such as 9-benzyl-8-hydroxy-2-(2-methoxyethoxy)adenine, methyladenosine, 8-Aza-7-deaza-A, 7-deaza-A, Vidarabine, 2-Aminoadenosine, N1-methyladenosine, 8-Azaadenosine, 5-Iodotubercidin, and N1-Me-dG. In some embodiments, the sugar moiety S′ in (III) is a sugar derivative as defined for Formula II.

In certain embodiments of the invention, the immune regulatory nucleic acid comprises a nucleic acid sequence containing at least one B-L-deoxynucleoside or 3′-deoxynucleoside.

The sequences of specific oligonucleotide-based TLR antagonists containing a modified immune stimulatory motif within these general structures used in the present study include, but are not limited to, COMPOUNDs/SEQ ID NOs 2-6 shown in Table 4.

TABLE 4

| COMPOUND/ | |
|---|---|
| SEQ ID NO: | Sequence |
| 1 | 5′-CTATCTGACGTTCTCTGT-3′ |
| | (TLR agonist control) |
| 2 | 5′-CTATCTGACG¹TTCTCTGT-3′ |
| 3 | 5′-CTATCTGAC¹GTTCTCTGT-3′ |
| 4 | 5′-CTATCTGAC²GTTCTCTGT-3′ |
| 5 | 5′-CTATCTGAC³GTTCTCTGT-3′ |
| 6 | 5′-CTATCTGAC²G¹TTCTCTGT-3′ |
| 7 | 5′-CTATCTGAC²CTTCTCTGT-3′ |
| | (inactive control oligonucleotide) |
| 8 | 5′-CTATCTG¹A*CCTTCTCTGT-3′ |
| | (inactive control oligonucleotide) |
| 9 | 5′-TGTC²GTTCT-X-TCTTGC²TGT-5′ |
| 10 | 5′-TGTC¹GTTCT-X-TCTTGC¹TGT-5′ |
| 11 | 5′-TGTCG¹TTCT-X-TCTTG¹CTGT-5′ |
| 12 | 5′-TCTGACG²1TTCT-X-TCTTG²CAGTCT-5′ |
| | (TLR agonist control) |

C¹ = 2′-O-methyl-C; C² = 5-methyl-dC; C³ =2′-O-methyl-5-methyl-C;
G¹ = 2′-O-methyl-G; G¹ = 7-deaza-dG; A* = 2′-O-methyl-A; 1,2,3-Propanetriol

In some embodiments, the oligonucleotide-based TLR antagonists containing a modified immune stimulatory motif each have from about 6 to about 35 nucleoside residues, preferably from about 9 to about 30 nucleoside residues, more preferably from about 11 to about 23 nucleoside residues. In some embodiments, the oligonucleotide-based TLR antagonist containing a modified immune stimulatory motif has from about 6 to about 18 nucleoside residues.

In a second aspect, the invention provides pharmaceutical formulations comprising one or more oligonucleotide-based TLR antagonist containing a modified immune stimulatory motif according to the invention and a physiologically acceptable carrier.

In a third aspect, the invention provides methods for inhibiting or suppressing TLR-mediated induction of an immune response in a mammal, such methods comprising administering to the mammal one or more oligonucleotide-based TLR antagonist containing a modified immune stimulatory motif according to the invention. In preferred embodiments, the oligonucleotide-based TLR antagonist containing a modified immune stimulatory motif is administered to a mammal in need of immune suppression.

According to this aspect of the invention, an oligonucleotide-based TLR antagonist containing a modified immune stimulatory motif is capable of suppressing a TLR-based immune response to a further TLR ligand or TLR agonist. The activation of a TLR-based immune response by a TLR agonist or TLR ligand (e.g. an immune modulatory oligonucleotide or bacterial DNA or viral RNA) can be suppressed/inhibited by the simultaneous, pre- or post-administration of an oligonucleotide-based TLR antagonist containing a modified immune stimulatory motif, and such suppression/inhibition may be maintained for an extended period of time (e.g. days) after administration. This beneficial property of the current invention has a unique advantage for the prevention and/or treatment of a disease or disorder. For example, application of certain TLR-agonists in the course of treating the disease may cause unwanted immune stimulation that an oligonucleotide-based TLR antagonist containing a modified immune stimulatory motif could suppress/inhibit. Administration of the oligonucleotide-based TLR antagonist containing a modified immune stimulatory motif simultaneously, pre and/or post administration of the TLR-agonist may allow therapeutic benefits from the TLR-agonist while suppressing/inhibiting the unwanted side effect(s). Additionally, pre-administration of an oligonucleotide-based TLR antagonist containing a modified immune stimulatory motif could prevent an immune response (e.g., allergic reaction) to a subsequent or later challenge by a TLR-agonist or ligand.

In the methods according to the invention, administration of an oligonucleotide-based TLR antagonist containing a modified immune stimulatory motif can be by any suitable route, including, without limitation, parenteral, mucosal delivery, oral, sublingual, transdermal, topical, inhalation, intranasal, aerosol, intraocular, intratracheal, intrarectal, vaginal, by gene gun, dermal patch or in eye drop or mouthwash form. Administration of the therapeutic compositions of oligonucleotide-based TLR antagonists containing a modified immune stimulatory motif can be carried out using known procedures at dosages and for periods of time effective to ameloriate or reduce symptoms or surrogate markers of the disease. When administered systemically, the therapeutic composition is preferably administered at a sufficient dosage to attain a blood level of oligonucleotide-based TLR antagonist containing a modified immune stimulatory motif from about 0.0001 micromolar to about 10 micromolar. For localized administration, much lower concentrations than this may be effective, and much higher concentrations may be tolerated. Preferably, a total dosage of oligonucleotide-based TLR antagonist containing a modified immune stimulatory motif ranges from about 0.001 mg per patient per day to about 200 mg per kg body weight per day. In certain preferred embodiments, the dosage of oligonucleotide-based TLR antagonist containing a modified immune stimulatory motif is 0.08, 0.16, 0.24, 0.32, 0.40, 0.48, 0.56 or 0.64 mg/kg. It may be desirable to administer simultaneously, or sequentially a therapeutically effective amount of one or more of the therapeutic compositions of the invention to an individual as a single treatment episode. In further embodiments, it may be desirable to administer the oligonucleotide-based TLR antagonist containing a modified immune stimulatory motif at regular intervals, including but not limited to daily, twice a week, weekly, twice a month or monthly.

The methods according to this aspect of the invention are useful for model studies of the immune system. The methods are also useful for the prophylactic or therapeutic treatment of human or animal disease. For example, the methods are useful for pediatric and veterinary vaccine applications.

In a fourth aspect, the invention provides methods for therapeutically treating a patient having a disease or disorder, such methods comprising administering to the patient an oligonucleotide-based TLR antagonist containing a modified immune stimulatory motif according to the invention. In various embodiments, the disease or disorder to be treated is cancer, an autoimmune disorder, airway inflammation, inflammatory disorders, infectious disease, malaria, Lyme disease, ocular infections, conjunctivitis, skin disorders, psoriasis, scleroderma, cardiovascular disease, atherosclerosis, chronic fatigue syndrome, sarcoidosis, transplant rejection, allergy, asthma or inflammation caused by a pathogen. Administration is carried out as described for the third aspect of the invention.

In a fifth aspect, the invention provides methods for preventing a disease or disorder, such methods comprising administering to a patient at risk for developing the disease or disorder one or more oligonucleotide-based TLR antagonists containing a modified immune stimulatory motif according to the invention. A patient is considered to be at risk of a disease or disorder if the patient has been exposed to an etiologic agent of such disease or disorder. In various embodiments, the disease or disorder to be prevented is cancer, an autoimmune disorder, airway inflammation, inflammatory disorders, infectious disease, malaria, Lyme disease, ocular infections, conjunctivitis, skin disorders, psoriasis, scleroderma, cardiovascular disease, atherosclerosis, chronic fatigue syndrome, sarcoidosis, transplant rejection, allergy, asthma or a inflammation caused by a pathogen. Pathogens include bacteria, parasites, fungi, viruses, viroids and prions. Preferred viruses include but are not limited to DNA or RNA virus. Administration is carried out as described for the third aspect of the invention.

In any of the methods according to the invention, the oligonucleotide-based TLR antagonist containing a modified immune stimulatory motif can be administered in combination with any other agent useful for treating the disease or condition that does not diminish the immune modulatory effect of the oligonucleotide-based TLR antagonist containing a modified immune stimulatory motif. In any of the methods according to the invention, the agent useful for treating the disease or condition includes, but is not limited to, one or more vaccines, antigens, antibodies, cytotoxic agents, allergens, antibiotics, antisense oligonucleotides, TLR agonist, TLR antagonist, peptides, proteins, gene therapy vectors, DNA vaccines, adjuvants, antiviral agents, antimalarial drugs (for example chloroquine, hydroxychloroquine, and immune suppressive drugs) or kinase inhibitors to enhance the specificity or magnitude of the immune response, or co-stimulatory molecules such as cytokines, chemokines, protein ligands, trans-activating factors, peptides and peptides comprising modified amino acids. For example, in the treatment of cancer, it is contemplated that the oligonucleotide-based TLR antagonist containing a modified immune stimulatory motif may be administered in combination with one or more chemotherapeutic compound, targeted therapeutic agent and/or monoclonal antibody. Alternatively, the agent can include DNA vectors encoding for antigen or allergen. In these embodiments, the oligonucleotide-based TLR antagonists containing a modified immune stimulatory motif of the invention can variously act to produce direct immune modulatory effects.

The following examples are intended to further illustrate certain exemplary embodiments of the invention and are not intended to limit the scope of the invention. For example, representative TLR-ligands are shown in the following examples, but do not limit the scope of ligands to which the oligonucleotide-based TLR antagonists containing a modified immune stimulatory motif of the invention act as antagonists.

### Example 1

### Synthesis of Oligonucleotides Containing Immune Regulatory Moieties

All oligonucleotide-based TLR antagonists containing a modified immune stimulatory motif and control oligonucleotides were synthesized according to standard procedures (see e.g. U.S. Pat. No. 7,276,489).

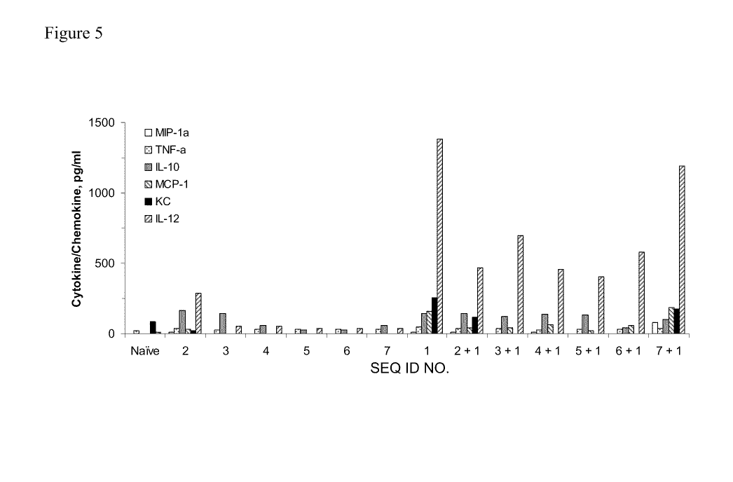

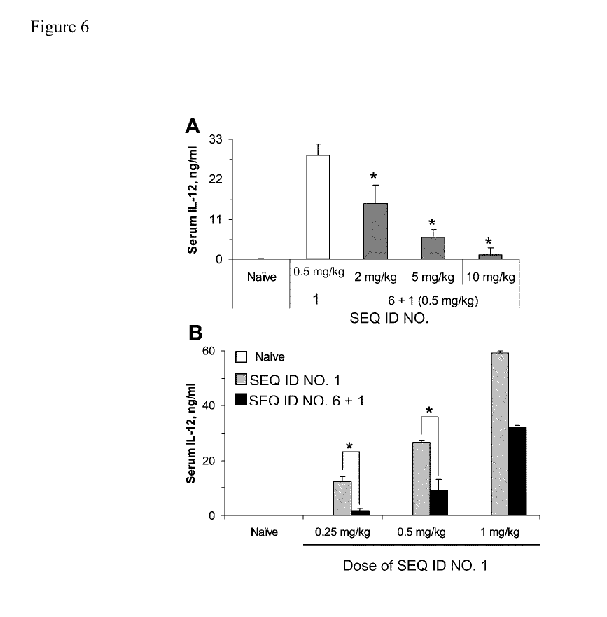

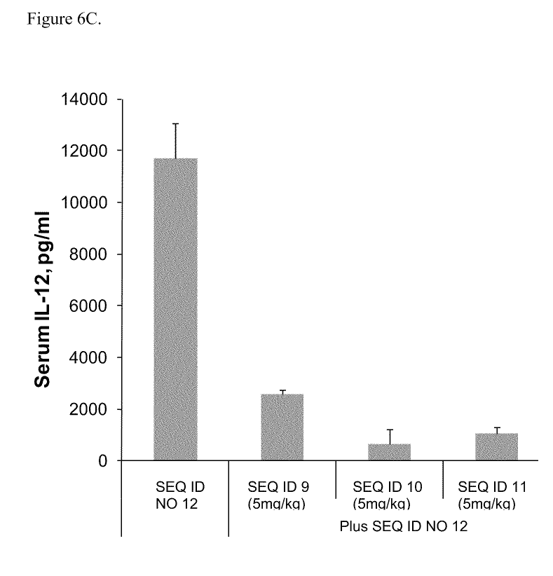

Oligonucleotides were synthesized on a 1 μM scale using an automated DNA synthesizer (Expedite 8909; PerSeptive Biosystems, Framingham, Mass.), following standard linear synthesis or parallel synthesis procedures (see e.g. FIGS. 5 and 6 of U.S. Pat. No. 7,276,489). All oligonucleotide-based TLR antagonists containing a modified immune stimulatory motif were characterized by capillary gel electrophoresis (CGE) or denaturing polyacrylamide gel electrophoresis (PAGE) and MALDI-TOF mass spectrometry (Waters MALDI microMX mass spectrometer) for purity and molecular mass, respectively. The purity of full-length oligonucleotides ranged from 95-99% with the remainder found to lack one or two nucleotides by HPLC, CGE, and/or denaturing PAGE. All oligonucleotide-based TLR antagonists containing a modified immune stimulatory motif contained <0.075 EU/mg of endotoxin by the Limulus assay (Bio-Whittaker).

### Example 2

### Inhibition of TLR9 Stimulation

### HEK293 Cells

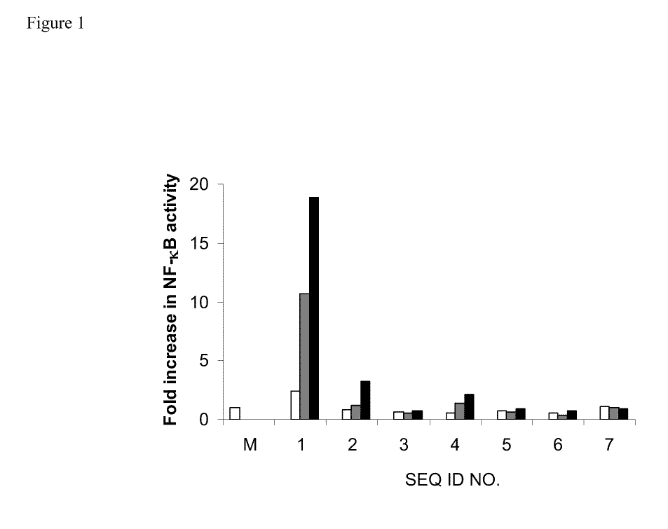

HEK293 cells stably expressing TLR9 (Invivogen) were transiently transfected with reporter gene, Seap, (Invivogen) for 6 hr. Cells were treated with 0.5 μg/ml of control TLR9 agonist (SEQ ID NO 1) alone and with exemplary oligonucleotide-based TLR antagonists containing a modified immune stimulatory motif (SEQ ID NOs 2-6) at 0.1 μg/ml, 0.3 μg/ml or 1.0 μg/ml or negative control (SEQ ID NO 7) alone for 18 hr. TLR9-dependent reporter gene, NF-κB, expression was determined according to the manufacturer's protocol (Invivogen) and the results are expressed as fold increase in NF-κB activity. The results are shown in FIG. 1.

### J774 Cells

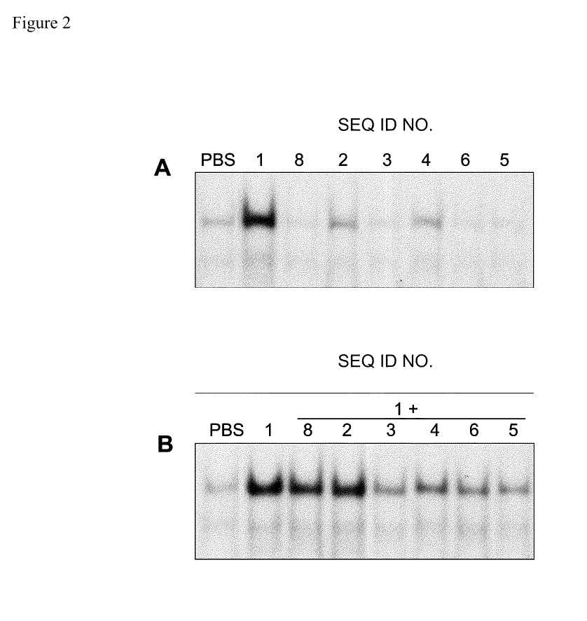

Murine J774 macrophage cells (American Type Culture Collection, Rockville, Md.) were cultured in Dulbecco's modified Eagle's medium supplemented with 10% (v/v) fetal bovine serum (FBS) and antibiotics (100 IU/ml penicillin G/100 μg/ml streptomycin). J774 cells were plated at a density of 5×10⁶ cells/well in six-well plates, treated with 0.5 μg/ml of control TLR9 agonist (SEQ ID NO 1) alone or with 2.5 μg/ml of exemplary oligonucleotide-based TLR antagonists containing a modified immune stimulatory motif (SEQ ID NOs 2-6) or a negative control oliginucleotide (SEQ ID NOs 7-8) for 1 hr, and nuclear extracts were prepared and analyzed for NF-κB activation by native polyacrylamide gels. Gels were dried and exposed to HyBlot CL autoradiography films at −70° C. Films were scanned and the images were processed using Adobe imaging software. The results are shown in FIG. 2.

### C57BL/6 Mouse Spleen Cells—1

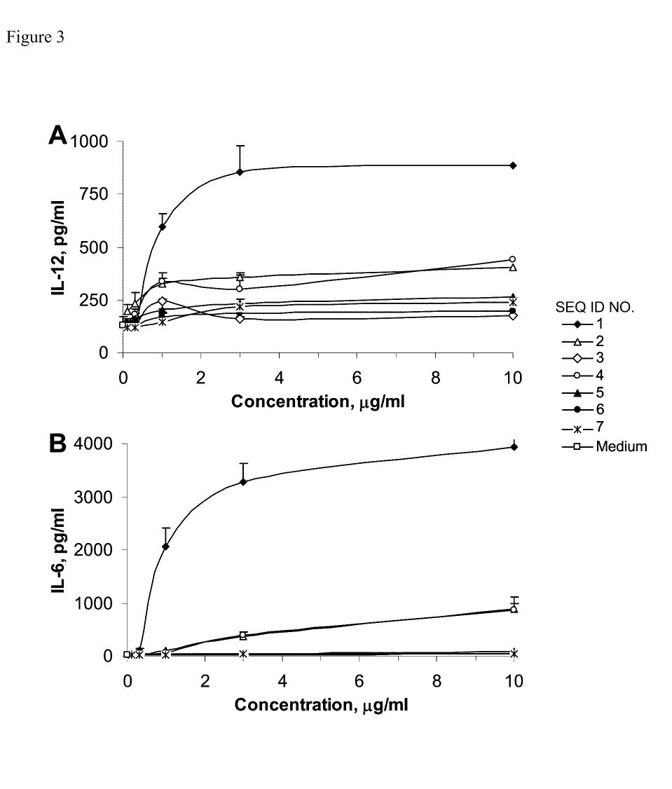

Spleen cells from 4- to 8-week old C57BL/6 mice were cultured in RPMI complete medium. Mouse spleen cells were plated in 24-well dishes using 5×10⁶ cells/ml, treated with increasing concentrations of control TLR9 agonist (SEQ ID NO 1), exemplary oligonucleotide-based TLR antagonists containing a modified immune stimulatory motif (SEQ ID NOs 2-6) or a negative control oligonucleotide (SEQ ID NOs 7-8) dissolved in TE buffer (10 mM Tris-HCl, pH 7.5, 1 mM EDTA), and incubated at 37° C. for 24 hr (dark bars). Following incubation, the supernatants were collected and the secretion of IL-12 and IL-6 in cell culture supernatants was measured by sandwich ELISA. Data are shown in FIGS. 3A and 3B and are representative of at least three independent experiments.

### C57BL/6 Mouse Spleen Cells—2

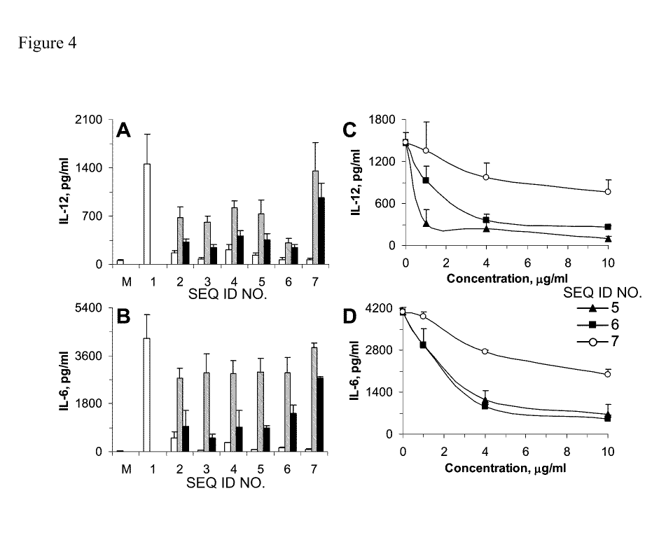

Spleen cells from 4- to 8-week old C57BL/6 mice were cultured in RPMI complete medium. Mouse spleen cells were plated in 24-well dishes using 5×10⁶ cells/ml, treated with 1 μg/ml control TLR9 agonist (SEQ ID NO 1) alone or with 4 μg/ml of exemplary oligonucleotide-based TLR antagonists containing a modified immune stimulatory motif (SEQ ID NOs 2-6) dissolved in TE buffer (10 mM Tris-HCl, pH 7.5, 1 mM EDTA), and incubated at 37° C. for 24 hr (dark bars). As a control, 4 μg/ml of exemplary oligonucleotide-based TLR antagonists containing a modified immune stimulatory motif (SEQ ID NOs 2-6) were incubated with the spleen cells in the absence of the control TLR9 agonist (white bars). Following incubation, the supernatants were collected and the secretion of IL-12 and IL-6 in cell culture supernatants was measured by sandwich ELISA. Data are shown in FIGS. 4A and 4B and are representative of at least three independent experiments.

### C57BL/6 Mouse Spleen Cells—3

Spleen cells from 4- to 8-week old C57BL/6 mice were cultured in RPMI complete medium. Mouse spleen cells were plated in 24-well dishes using 5×10⁶ cells/ml, treated with 1 μg/ml control TLR9 agonist (SEQ ID NO 1) alone or with increasing concentrations of exemplary oligonucleotide-based TLR antagonists containing a modified immune stimulatory motif (SEQ ID NOs 2-6) dissolved in TE buffer (10 mM Tris-HCl, pH 7.5, 1 mM EDTA), and incubated at 37° C. for 24 hr. Following incubation, the supernatants were collected and the secretion of IL-12 and IL-6 in cell culture supernatants was measured by sandwich ELISA. Data are shown in FIGS. 4C and 4D and are representative of at least three independent experiments.

### Example 3

### In Vivo Inhibition of TLR Activity by Oligonucleotide-Based TLR Antagonists containing a Modified Immune Stimulatory Motif

Female C57BL/6 mice, five to six weeks old, (n=3) were injected subcutaneously with exemplary oligonucleotide-based TLR antagonists containing a modified immune stimulatory motif (SEQ ID NOs 2-6). For acute administration studies, C57BL/6 mice were injected with exemplary oligonucleotide-based TLR antagonists containing a modified immune stimulatory motif at 2 mg/kg subcutaneously in the right flank. For inhibition experiments, 2 mg/kg of exemplary oligonucleotide-based TLR antagonists containing a modified immune stimulatory motif were administered in the right flank and 24 hr later 0.5 mg/kg control TLR9 agonist (SEQ ID NO 1) was administered subcutaneously in the left flank. Blood was collected by retro-orbital bleeding 2 hr after administration of the control TLR9 agonist and serum cytokines and chemokines were measured.

Serum samples from in vivo experiments were assayed using multiplex luminescent beads (Mouse cytokine twenty-plex, Invitrogen, Camarillo, Calif.) according to the manufacturer's instructions and analyzed with a Luminex 100/200 instrument. Fluorescence intensity was transformed into cytokine concentration using StarStation software (Applied Cytometry Systems). Some serum samples were analyzed for IL-12 levels by ELISA. Data shown in FIG. 5 are representative of two independent experiments. * Indicates p<0.05.

### Example 4

### In Vivo Inhibition of TLR Activity by Oligonucleotide-Based TLR Antagonists Containing a Modified Immune Stimulatory Motif

Female C57BL/6 mice, five to six weeks old, (n=3) were injected subcutaneously with 2, 5 or 10 mg/kg of exemplary oligonucleotide-based TLR antagonist containing a modified immune stimulatory motif (SEQ ID NO. 6) in the right flank and twenty-four hours later with 0.5 mg/kg or 0.25 mg/kg control TLR9 agonist (SEQ ID NO 1 or SEQ ID NO 12) in the left flank. Blood was collected by retro-orbital bleeding 2 hr after administration of the control TLR9 agonist and serum IL-12 concentration was measured by ELISA. Data shown in FIGS. 6A, 6B, and 6C are representative of two independent experiments. * Indicates p<0.05.

### Example 5

### In Vivo Inhibition of TLR Activity by Oligonucleotide-Based TLR Antagonists Containing a Modified Immune Stimulatory Motif

Female C57BL/6 mice, five to six weeks old, (n=3) were injected subcutaneously with 10 mg/kg of exemplary oligonucleotide-based TLR antagonist containing a modified immune stimulatory motif (SEQ ID NO. 6) in the right flank and 24 hr later injected in the left flank with 0.25, 0.5 or 1 mg/kg of control TLR9 agonist (SEQ ID NO 1). Blood was collected by retro-orbital bleeding 2 hr after administration of the control TLR9 agonist and serum IL-12 concentration was measured by ELISA. Data shown in FIG. 6B are for a representative experiment of two or more independent experiments. * Indicates p<0.05.

### Example 6

### Duration of TLR Inhibition by Oligonucleotide-Based TLR Antagonists Containing a Modified Immune Stimulatory Motif

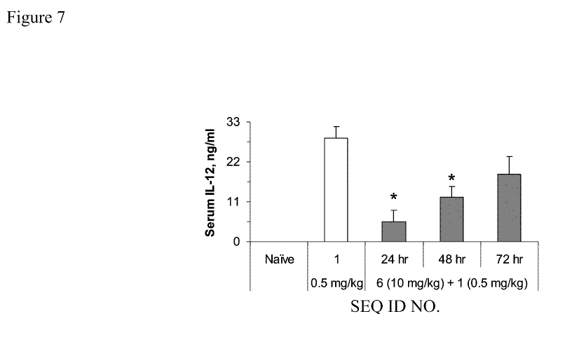

Female C57BL/6 mice were injected subcutaneously with 10 mg/kg of exemplary oligonucleotide-based TLR antagonist containing a modified immune stimulatory motif (SEQ ID NO. 6) in the right flank and at 24, 48 or 72 hours later injected in the left flank with 10 mg/kg control TLR9 agonist (SEQ ID NO. 1). Blood was collected by retro-orbital bleeding 2 hr after administration of the control TLR9 agonist and serum IL-12 concentration was measured by ELISA. Data shown in FIG. 7 are for a representative experiment of two or more independent experiments. * Indicates p<0.05.

### Example 7

### Specificity of TLR Inhibition by Oligonucleotide-Based TLR Antagonists Containing a Modified Immune Stimulatory Motif

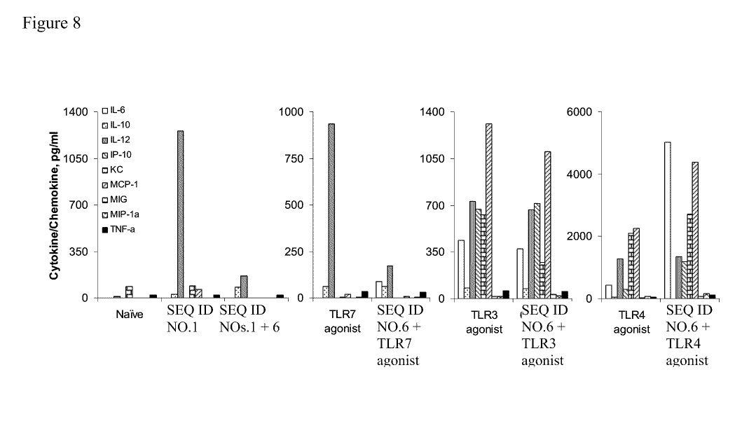

C57BL/6 mice were injected subcutaneously with 10 mg/kg exemplary oligonucleotide-based TLR antagonist containing a modified immune stimulatory motif (SEQ ID NO. 6) in the right flank and 24 hr later in the left flank with 0.5 mg/kg control TLR9 agonist (SEQ ID NO. 1), 10 mg/kg control TLR7 agonist (RNA-based compound), 25 mg/kg control TLR3 agonist (polyI.polyC) or 0.25 mg/kg control TLR4 agonist (LPS) was injected subcutaneously in the left flank. Blood was drawn 2 hr after agonist administration of the agonist and serum cytokine/chemokine levels were determined by luminex multiplex assay. Data shown in FIG. 8 are for a representative experiment of two or more independent experiments.

What is claimed is:

1. An oligonucleotide-based TLR antagonist containing a modified immune stimulatory motif comprising one or more modified immune stimulatory motifs, wherein CG is the modified immune stimulatory motif, wherein C is cytosine, or a pyrimidine nucleotide derivative selected from 5-methyl-dC, 2′-O-substituted-C, 2′-O-methyl-C, 2′-O-methoxyethoxy-C, 2′-O-methoxyethyl-5-methyl-C, and 2′-O-methyl-5-methyl-C, and G is guanosine or a purine nucleotide derivative selected from 2′-O-substituted-G, 2′-O-methyl-G, and 2′-O-methoxyethoxy-G; provided that at least one C and/or G of the modified immune stimulatory motif is a specified nucleotide derivative; wherein the modified immune stimulatory motif would be immune stimulatory but for the nucleotide derivative.

2. The oligonucleotide-based TLR antagonist according to claim 1 comprising at least two oligonucleotides linked through 3′, 2′, or 5′ attachments.

3. The antagonist according to claim 2 wherein the oligonucleotides are linked directly to each other at their 3′, 2′, or 5′ ends.

4. The antagonist according to claim 2 wherein the 3′, 2′, or 5′ ends of the oligonucleotide are linked to a non-nucleotidic linker.

5. The antagonist according to claim 4, wherein the linker is selected from the group consisting of Glycerol (1,2,3-Propanetriol), 1,2,4, Butanetriol, 2-(hydroxymethyl)1,4-butanediol, 1,3,5-Pentanetriol, 1,1,1-Tris(hydroxymethyl)ethane,1,1,1-Tris(hydroxymethyl)nitromethane, 1,1,1-Tris(hydroxymethyl)propane, 1,2,6-Methyl-1,3,5-pentanetriol, 1,2,3-Heptanetriol, 2-Amino-2-(hydroxymethyl)-1,3-propanediol, N[Tris(hydroxymethyl)methyl]acrylamide, cis-1,3,5-Cyclohexanetriol, Cis-1,3,5-Tri(hydroxymethyl)cyclohexane, 1,3,5-Trihydroxyl-benzene, 3,5-Di(hydroxymethyl)benzene, 1,3-Di(hydroxyethoxy)-2-hydroxyl-propane, 1,3-Di(hydroxypropoxy)-2-hydroxyl-propane, 2-Deoxy-D-ribose, 1,2,4-Trihydroxyl-benzene, D-Galactoal, 1,6-anhydro-β-D-Glucose, 1,3,5-Tris(2-hydroxyethyl)-Cyanuric acid, Gallic acid, 3,5,7-Trihydroxyflavone, 4,6-Nitropyrogallol, Ethylene glycol, 1,3-Propanediol, 1,2-Propanediol, 1,4-Butanediol, 1,3-Butanediol, 2,3-Butanediol, 1,4-Butanediol, 1,5-Pentanediol, 2,4-Pentanediol, 1,6-Hexanediol, 1,2-Hexanediol, 1,5-Hexanediol, 2,5-Hexanediol, 1,7-Heptanediol, 1,8-Octanediol, 1,2-Octanediol, 1,9-Nonanediol, 1,12-Dodecanediol, Triethylene glycol, Tetraethylene glycol, Hexaethylene glycol, 2-(1-Aminopropyl)-1,3-propanediol, and 1,2-Dideoxyribose.

6. A pharmaceutical composition comprising the oligonucleotide according to claim 1 and a pharmaceutically acceptable carrier.

7. A method for modifying a TLR-stimulating oligonucleotide comprising an immune stimulatory motif, the method comprising incorporating chemical modifications into the immune stimulatory motif, wherein CG is the immune stimulatory motif and the chemical modification is selected from 5-methyl-dC, 2′-O-substituted-C, 2′-O-methyl-C, 2′-O-methoxyethoxy-C, 2′-O-methoxyethyl-5-methyl-C, 2′-O-methyl-5-methyl-C, 2′-O-substituted-G, 2′-O-methyl-G, and 2′-O-methoxyethoxy-G.

8. A method for modifying a TLR-stimulating oligonucleotide comprising an immune stimulatory motif, the method comprising incorporating chemical modifications into the immune stimulatory motif and/or to a sequence flanking the immune stimulatory motif, wherein CG is the immune stimulatory motif and the chemical modification is selected from 5-methyl-dC, 2′-O-substituted-C, 2′-O-methyl-C, 2′-O-methoxyethoxy-C, 2′-O-methoxyethyl-5-methyl-C, 2′-O-methyl-5-methyl-C, 2′-O-substituted-G, 2′-O-methyl-G, and/or 2′-O-methoxyethoxy-G.

9. A method for inhibiting a TLR7- or TLR9-mediated immune response in a mammal comprising administering to a mammal an oligonucleotide-based TLR antagonist according to claim 1.

10. The method according to claim 9, wherein the route of administration is parenteral, mucosal delivery, oral, sublingual, transdermal, topical, inhalation, intranasal, aerosol, intraocular, intratracheal, intrarectal, vaginal, by gene gun, dermal patch or in eye drop or mouthwash form.

11. A method for therapeutically treating a disease or disorder mediated by a TLR comprising administering to a mammal having the disease or disorder a therapeutically effective amount of an oligonucleotide-based TLR antagonist according to claim 1.

12. The method according to claim 11, wherein the route of administration is parenteral, mucosal delivery, oral, sublingual, transdermal, topical, inhalation, intranasal, aerosol, intraocular, intratracheal, intrarectal, vaginal, by gene gun, dermal patch or in eye drop or mouthwash form.

13. A method for prophylactically preventing a disease or disorder mediated by a TLR comprising administering to a mammal at risk of developing the disease or disorder a prophylactically effective amount of an oligonucleotide-based TLR antagonist according to claim 1.

14. The method according to claim 13, wherein the route of administration is parenteral, mucosal delivery, oral, sublingual, transdermal, topical, inhalation, intranasal, aerosol, intraocular, intratracheal, intrarectal, vaginal, by gene gun, dermal patch or in eye drop or mouthwash form.

15. A method for preventing cancer, an autoimmune disorder, airway inflammation, inflammatory disorders, infectious disease, skin disorders, allergy, asthma or a disease caused by a pathogen in a vertebrate, such method comprising administering to the vertebrate a TLR-based antagonist according to claim 1 in a pharmaceutically effective amount.

16. The method according to claim 15, wherein the TLR-based antagonist is administered in combination with one or more vaccines, antigens, antibodies, cytotoxic agents, allergens, antibiotics, antisense oligonucleotides, TLR agonists, TLR antagonists, peptides, proteins, gene therapy vectors, DNA vaccines, adjuvants or co-stimulatory molecules.

17. The method according to claim 15, wherein the route of administration is parenteral, mucosal delivery, oral, sublingual, transdermal, topical, inhalation, intranasal, aerosol, intraocular, intratracheal, intrarectal, vaginal, by gene gun, dermal patch or in eye drop or mouthwash form.


SEQUENCE LISTING (12 sequences)

SEQ ID NO: 1 - DNA, 18 bp, Artificial Sequence
  Description of Artificial Sequence Synthetic oligonucleotide
ctatctgacgttctctgt

SEQ ID NO: 2 - DNA, 18 bp, Artificial Sequence
  Description of Artificial Sequence Synthetic oligonucleotide
ctatctgacgttctctgt

SEQ ID NO: 3 - DNA, 18 bp, Artificial Sequence
  Description of Artificial Sequence Synthetic oligonucleotide
ctatctgacgttctctgt

SEQ ID NO: 4 - DNA, 18 bp, Artificial Sequence
  Description of Artificial Sequence Synthetic oligonucleotide
ctatctgacgttctctgt

SEQ ID NO: 5 - DNA, 18 bp, Artificial Sequence
  Description of Artificial Sequence Synthetic oligonucleotide
ctatctgacgttctctgt

SEQ ID NO: 6 - DNA, 18 bp, Artificial Sequence
  Description of Artificial Sequence Synthetic oligonucleotide
ctatctgacgttctctgt

SEQ ID NO: 7 - DNA, 18 bp, Artificial Sequence
  Description of Artificial Sequence Synthetic oligonucleotide
ctatctgaccttctctgt

SEQ ID NO: 8 - DNA, 18 bp, Artificial Sequence
  Description of Artificial Sequence Synthetic oligonucleotide
ctatctgaccttctctgt

SEQ ID NO: 9 - DNA, 9 bp, Artificial Sequence
  Description of Artificial Sequence Synthetic oligonucleotide
tgtcgttct

SEQ ID NO: 10 - DNA, 9 bp, Artificial Sequence
  Description of Artificial Sequence Synthetic oligonucleotide
tgtcgttct

SEQ ID NO: 11 - DNA, 9 bp, Artificial Sequence
  Description of Artificial Sequence Synthetic oligonucleotide
tgtcgttct

SEQ ID NO: 12 - DNA, 11 bp, Artificial Sequence
  Description of Artificial Sequence Synthetic oligonucleotide
tctgacgttct